(19) United States
(12) Patent Application Publication
(10) Pub. No.: US 2008/0312513 A1
(43) Pub. Date: Dec. 18, 2008

(54) Neurosurgical Candidate Selection Tool

(76) Inventors: Ely Simon, Bayside, NY (US); Glen M. Doniger, Houston, TX (US); Michael S. Okun, Gainesville, FL (US); Hubert H. Fernandez, Gainesville, FL (US); Kelly D. Foote, Gainesville, FL (US)

Correspondence Address: Weingarten Schurgin Gagnebin & Lebovici, LLP;ABBO, Ten Post Office Square, BOSTON, MA, 02109, US

(21) Appl. No.: 11/909,222

(22) PCT Filed: Mar. 21, 2006
(86) PCT No.: PCT/IL2006/000360

§ 371 Date: May 22, 2008

Related U.S. Application Data
(60) Provisional application No. 60/663,232, filed on Mar. 21, 2005.

(51) Int. Cl. A61B 5/00; G06N 5/02

(52) U.S. Cl. 600/300; 706/46

(57) ABSTRACT

A system and method for neurosurgery candidacy assessment includes multiple data sources, wherein results of tests from at least some of the multiple data sources are integrated. A neurosurgery candidacy assessment report including a recommendation regarding candidacy for neurosurgery is provided based on the integrated results. The multiple data sources may include cognitive tests, a background data source, a medical data source, an anxiety/depression data source, and a motor skills data source. The medical data source may include a FLASQ-PD questionnaire.

This application claims priority from U.S. Provisional Patent Application Ser. No. 60/663,232, filed on Mar. 21, 2005, entitled “Neurosurgical Candidate Selection Tool”, incorporated by reference herein in its entirety.

### FIELD OF THE INVENTION

The present invention relates to systems and methods for standardizing the measuring, evaluating and reporting of neurological skills and candidacy for neurological surgery.

### BACKGROUND OF THE INVENTION

Many invasive procedures, particularly in the field of neurosurgery require a selection process to determine whether an individual would be a suitable candidate. Most often, a physician makes this determination based on a clinical examination and medical history. However, the determination is often subjective, particularly when clear guidelines are lacking.

An example of a procedure requiring selection of candidates is Deep Brain Stimulation (DBS), a surgical procedure used to treat symptoms primarily associated with Parkinson's disease (PD), such as tremor, rigidity, stiffness, slowed movement, and walking problems.

The surgical procedure involves implantation of a neurostimulator device—which is a battery operated device similar to a heart pacemaker. The neurostimulator device is designed to deliver electrical stimulation to the areas in the brain which control movement. There are three components of the device, including the neurostimulator (battery component), an electrode component, and an extension. The neurostimulator is generally implanted under the skin near the collarbone, or elsewhere in the chest or abdomen. The electrode component is implanted in the brain, in an area predetermined for the individual on the basis of magnetic resonance imaging (MRI) or computed tomography (CT) scanning. The targeted area is generally the thalamus. The extension is an insulated wire connecting the electrode to the neurostimulator, and is passed through the shoulder, head and neck. Impulses are sent from the neurostimulator, along the extension wire, and into the brain via the electrode. The impulses block electrical signals from the targeted area of the brain.

Candidacy for DBS is generally determined by the physician, based on various factors, including cognitive function status, whether the Parkinson's is idiopathic, how the patient responds to certain medications, age and other factors. There are currently no existing computerized standardized screening tools to aid the physician in the decision-making process.

It would be useful to have a standardized selection tool for use in determining candidacy for neurosurgical procedures such as DBS.

### SUMMARY OF THE INVENTION

According to one aspect of the invention, there is provided a computerized system for evaluating candidacy of a patient for neurosurgery. The system includes a cognitive testing data source, including at least one cognitive test for testing at least one cognitive domain of a subject, the test providing cognitive data for the cognitive domain, at least one additional data source providing additional data, a processor configured to integrate the cognitive data and the additional data, and a reporting module in communication with the processor and configured to provide a neurosurgery candidacy recommendation based on the integrated data.

According to another aspect of the invention, there is provided a method of integrating results from various data sources. The method includes comparing first test results to a first test exclusion threshold and a first test inclusion threshold, designating the first test results as pass, fail, or inconclusive based on the comparison, comparing second test results to a second test fail threshold and a second test pass threshold, designating the second test results as pass, fail, or inconclusive based on the comparison, determining an overall number of passes, an overall number of fails and an overall number of inconclusive designations, integrating the overall numbers into a final score, and reporting a neurosurgery candidacy recommendation based on the integrated score, wherein the comparing, designating, reporting and integrating are done using a processor.

According to yet another aspect of the invention, there is provided a method of assessing neurosurgery candidacy of a subject. The method includes presenting stimuli for a cognitive test for measuring a cognitive domain, collecting responses to the stimuli, calculating an outcome measure based on the responses, collecting additional data from an additional data source, and calculating a unified score based on the outcome measure and the additional data source.

According to further features in embodiments of the invention, the additional data source may include multiple additional data sources, which may be selected from the group consisting of a background data source, a medical data source, an anxiety/depression data source, and a motor skills data source. The medical data source may include, for example, a FLASQ-PD questionnaire. The anxiety/depression data source may include, for example, a Zung Anxiety scale and/or a geriatric depression scale. The cognitive test may include multiple cognitive tests, and may include, for example, a test for information processing, a test for executive function, a test for attention, a test for motor skills, and a test for memory.

The candidacy recommendation may be a recommendation that the patient is a good surgical candidate, a recommendation that the patient is not a good surgical candidate for certain reasons, a recommendation that the patient might be a good surgical candidate but that further evaluation is warranted, or any other suitable recommendation.

In yet further features, the integrated data may include an index score and/or a composite score. The processor may include selectors, including a domain selector for selecting a cognitive domain and/or a test selector for selecting a cognitive test. The reporting module may include summaries of the cognitive data and the additional data, and a score for the integrated data, which may be depicted in graphical format.

According to further features, the comparing of first and second test results may include comparing cognitive test results to one or more of either background data source results, medical data source results, motor skills data source results and anxiety/depression data source results.

According to yet additional features, the unified score may in some embodiment be an index score or a composite score. An index score could be a combination of an outcome measure of a cognitive test and additional data, wherein the cognitive test and the additional data source are for measurement of the same cognitive domain. The index score may also be a combination of outcome measures from a particular test or from multiple tests in a particular cognitive domain. The composite score may be a combined score of an index score and an outcome measure, from two index scores, or from outcome measures and additional data directly.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of is conflict, the patent specification, including definitions, will control. In addition, the materials, methods, and examples are illustrative only and not intended to be limiting.

### BRIEF DESCRIPTION OF THE DRAWINGS

The above and further advantages of the present invention may be better understood by referring to the following description in conjunction with the accompanying drawings in which:

FIG. 1 is a schematic illustration of a system in accordance with embodiments of the present invention;

FIG. 2 is a schematic illustration of a cognitive testing data source;

FIG. 3 is a schematic illustration of a method of using the cognitive testing data source of FIG. 2 to compute cognitive testing scores;

FIG. 4 is a block diagram illustration showing the steps of the method of FIG. 3;

FIG. 5 is a schematic illustration of one specific example of the multi-layered collection of data generally depicted in the schematic illustration of FIG. 2;

FIG. 6 is a flow chart diagram illustration of the steps of a cognitive test in accordance with one embodiment of the present invention;

FIG. 7 is a flow chart diagram illustration of the steps of a finger tap test according to one embodiment of the present invention;

FIG. 8 is a pictorial sample illustration of a screen shot from a catch test in accordance with one embodiment of the present invention;

FIGS. 9A-9E are illustrations of a medical data source in accordance with one embodiment of the present invention;

FIG. 10 is an illustration of an anxiety data source, in accordance with one embodiment of the present invention;

FIG. 11 is an illustration of a depression data source, in accordance with one embodiment of the present invention;

FIG. 12 is a flow chart diagram illustration of a method of providing a designation for a particular test based on the results of that test; and

FIG. 13 is a flow chart diagram illustration of a method of integrating results from multiple tests from some or all of the data sources of the present invention, in accordance with one embodiment.

It will be appreciated that for simplicity and clarity of illustration, elements shown in the drawings have not necessarily been drawn accurately or to scale. For example, the dimensions of some of the elements may be exaggerated relative to other elements for clarity or several physical components may be included in one functional block or element. Further, where considered appropriate, reference numerals may be repeated among the drawings to indicate corresponding or analogous elements. Moreover, some of the blocks depicted in the drawings may be combined into a single function.

### DETAILED DESCRIPTION OF THE INVENTION

In the following detailed description, numerous specific details are set forth in order to provide a thorough understanding of the present invention. It will be understood by those of ordinary skill in the art that the present invention may be practiced without these specific details. In other instances, well-known methods, procedures, components and structures may not have been described in detail so as not to obscure the present invention.

The present invention is directed to a standardized neurosurgical candidate selection tool for determining candidacy for DBS and other surgical interventions.

A system and method for screening and evaluation of neurological function is described in U.S. Patent Publication Number 2005-0142524 to Simon et al., (referred to hereinafter as the '524 Publication) and is incorporated by reference herein in its entirety. In Simon et al., a system is disclosed which is designed to provide an initial view of cognitive function to a physician, prior to or concurrent with a clinical examination. The present application uses some of the components of the system disclosed in Simon et al., but specifically tailored for assessment of neurosurgical candidacy.

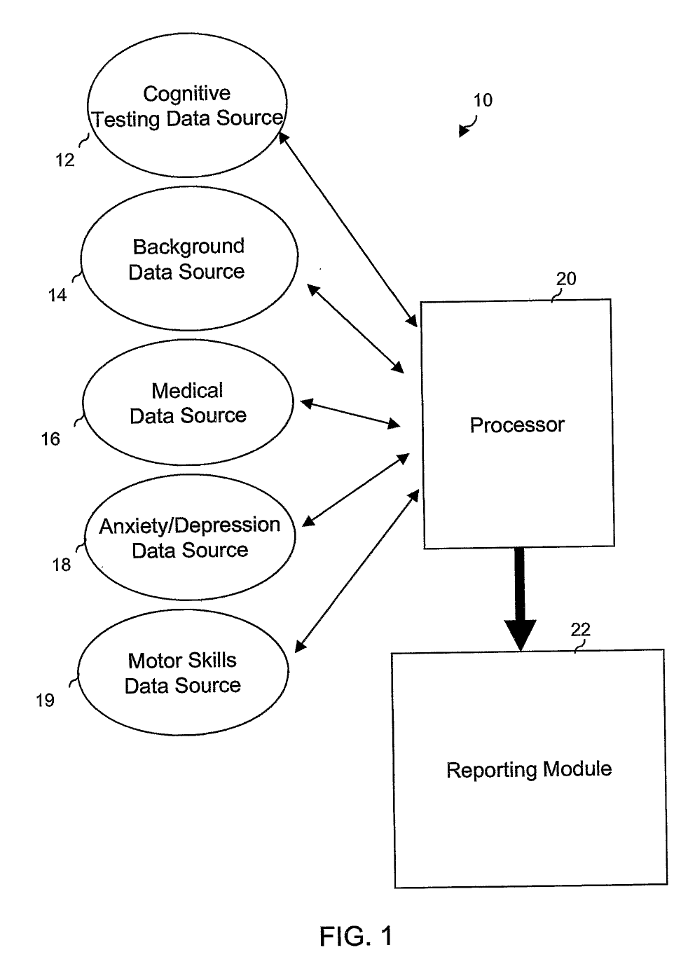

Reference is now made to FIG. 1, which is a schematic illustration of a system 10 in accordance with embodiments of the present invention. System 10 includes multiple data sources, including a cognitive testing data source 12, a background data source 14, a medical data source 16, an anxiety/depression data source 18, and a motor skills data source 19. System 10 further includes a data processor 20 for processing data received from some or all of data sources 12, 14, 16, 18, and 19, and a reporting module 22 for presenting processed data. System 10 is an interactive system, wherein data from any one of data sources 12, 14, 16, 18 and 19 may be used by processor 20 to determine output of the other data sources. For example, information received by processor 20 from medical data source 16 may be used to determine what data should be collected from cognitive testing data source 12. Alternatively, a combination of collected data from some of data sources 12, 14, 16, 18 and 19 may be used by processor 20 to determine output of the other data sources. Additionally, information received from any or all of data sources 12, 14, 16, 18 and 19, or from any combination thereof, may be selectively or non-selectively combined in various ways by processor 20, and sent in various formats to reporting module 22. For the purposes of the present invention, “tests” refers generally to any evaluation by any of data sources 12, 14, 16, 18 or 19.

### Cognitive Testing Data Source

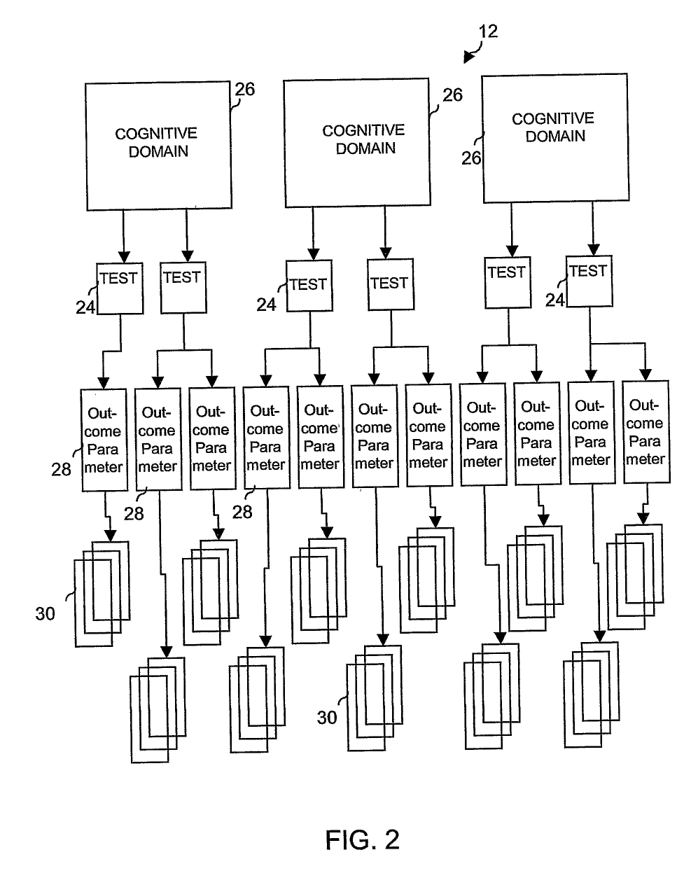

Reference is now made to FIG. 2, which is a schematic illustration of cognitive testing data source 12. As shown in FIG. 2, cognitive testing data source 12 is a system which may include one or more tests 24 for one or more cognitive domains 26. Cognitive domains 26 may include, for example, motor skills, memory, executive function, attention, information processing, general intelligence, motor planning, motor learning, emotional processing, useful visual fields, verbal skills, problem solving ability, or any other cognitive domain. Tests 24 for motor skills may include, for example, a finger tap test designed to assess speed of tapping and regularity of finger movement, and a catch test designed to assess hand/eye coordination, speed of movement, motor planning, and spatial perception. Tests 24 for memory may include, for example, a verbal memory test or a non-verbal memory test. Tests 24 for executive function may include, for example, a Stroop test and a Go/NoGo Inhibition Test. These tests are described more fully in US Patent Publication Number 2004-0167380, (referred to hereinafter as the '380 Publication), incorporated by reference herein in its entirety. The tests 24 of the present invention, however, are not limited to the ones listed above or the ones described in the '380 Publication. It should be readily apparent that many different cognitive tests may be used and are all within the scope of the invention.

Each test 24 may have one or more measurable outcome parameters 28, and each outcome parameter 28 has outcomes 30 obtained from user input in response to stimuli of tests 24. Multiple responses or outcomes 30 for each outcome parameter 28 may be collected, either sequentially, simultaneously, or over a period of time. Outcome parameters 28 may include, for example, response time, accuracy, performance level, learning curve, errors of commission, errors of omission, or any other relevant parameters. Thus, as will be described in greater detail hereinbelow, cognitive testing data source 12 may provide many layers of testing and data collection options.

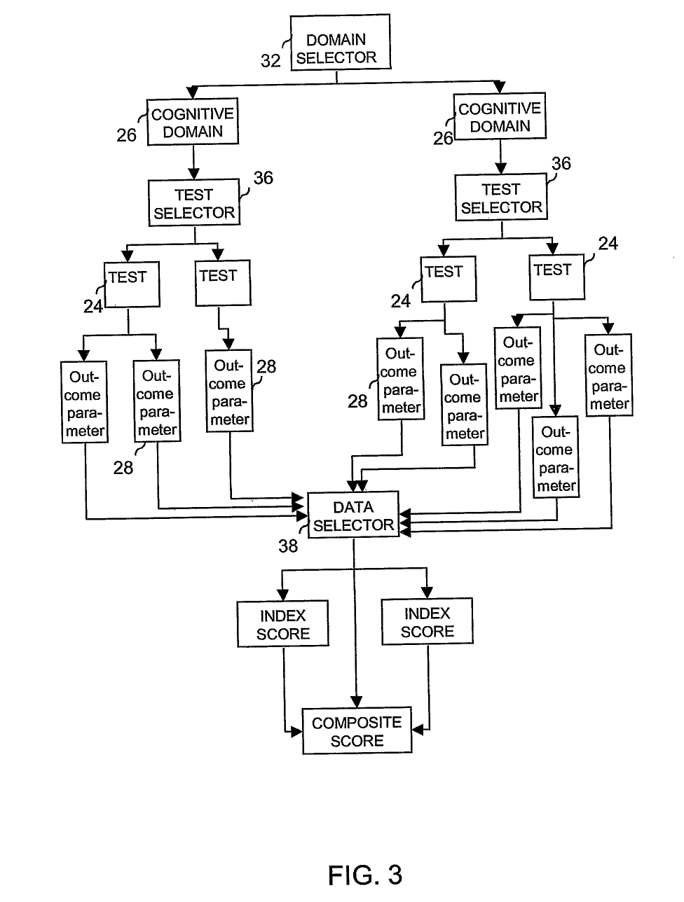

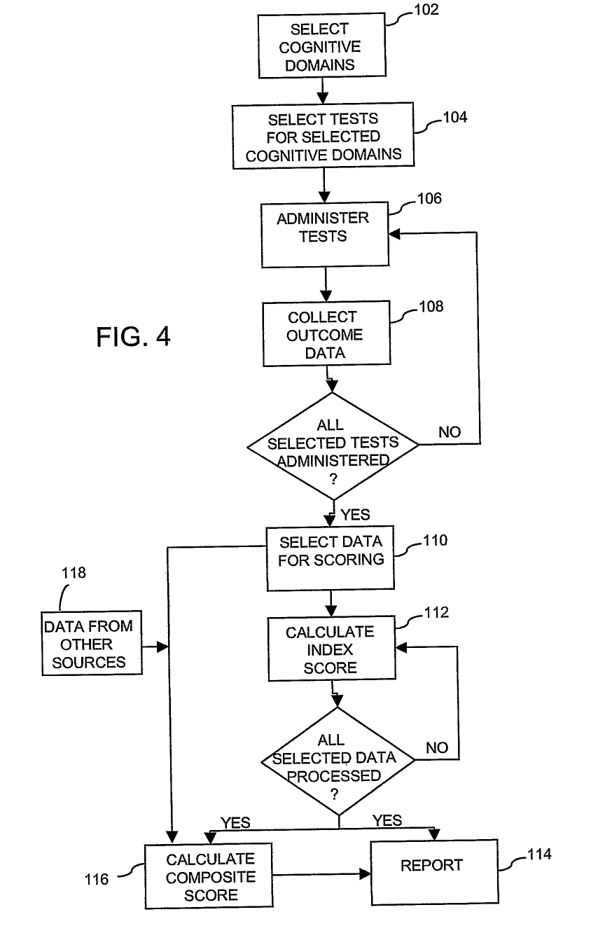

Reference is now made to FIGS. 3 and 4, which are schematic and block diagram illustrations, respectively, of a method of using cognitive testing data source 12 to compute cognitive testing scores for selected cognitive domains, for overall cognitive performance, and for an overall score or indication for neurosurgical candidacy. First, a domain selector 32 selects (step 102) cognitive domains 26 appropriate for the specific battery of tests. In one embodiment, domain selector 32 is an automated selector and may be part of processor 20 of system 10 depicted in FIG. 1. Selection of cognitive domains may be based on previously collected data from the same individual, background data from background data source 14, medical data from medical data source 16, known and/or published data in the field of neuropsychology or other related fields, known and/or published data regarding screening for neurosurgery, or input from a clinician or testing administrator. Alternatively, domain selector 32 may be a clinician or testing administrator, manually selecting specific cognitive domains 26 based on a clinical examination, patient status, or other information as listed above with respect to automated selection. This may be done, for example, by providing pre-packaged batteries focusing on specific domains. Alternatively, a “domain selection wizard” may help the clinician select the appropriate domains, based on interactive questions and responses. These can lead to a customized battery for a particular individual. Additionally, domain selection may be done after administration of some or all of the other elements of system 10, either automatically or manually based on initial results.

For each cognitive domain 26, a test selector 36 selects (step 104) tests 24. In one embodiment, test selector 36 is the same as domain selector 32. In another embodiment, test selector 36 is different from domain selector 32. For example, domain selector 32 may be a testing administrator while test selector 36 is an automated selector in processor 20. Alternatively, both domain selector 32 and test selector 36 may be automated selectors in processor 20, but may be comprised of different components within processor 20. Selection of tests for cognitive domains may be based on previously collected data from the same individual, background data from background data source 14, medical data from medical data source 16, known and/or published data in the field of neuropsychology or other related fields, known and/or published data regarding screening for neurosurgery, input from a clinician or testing administrator, clinical examination results, patient status, or any other known information. Processor 20 of system 10 then administers (step 106) a test 24 selected by test selector 36. Processor 20 collects (step 108) outcome data from each of the outcome parameters of the selected test. The steps of administering a selected test and collecting outcome data from outcome parameters of the selected test are repeated until all selected tests 24 for all selected cognitive domains 26 have been administered, and data has been collected from the selected and administered tests 24.

A data selector 38 may then select (step 110) data from all of the collected outcomes for processing and scoring. In one embodiment, data selector 36 is the same as domain selector 32 and/or test selector 36. In another embodiment, data selector 38 is different from either or both of domain selector 32 and test selector 36. For example, domain selector 32 may be a testing administrator while data selector 38 is an automated selector in processor 20. Alternatively, domain selector 32, test selector 36 and data selector 38 may be automated selectors in processor 20, but may be comprised of different components within processor 20. In some embodiments, data selector 38 is a pre-programmed selector, wherein for particular domains or tests, specific outcome measures will always be included in the calculation. Selection of data for processing may be based on previously collected data from the same individual, background data from background data source 14, medical data from medical data source 16, known and/or published data in the field of neuropsychology or other related fields, known and/or published data regarding screening for neurosurgery, input from a clinician or testing administrator, clinical examination results, patient status, or any other known information. In one embodiment, data selector 38 selects all of the collected data. In another embodiment, data selector 38 selects a portion of the collected data.

Processor 20 then calculates (step 112) index scores for the selected data and/or calculates (step 116) composite scores for the selected data. In one embodiment, index scores are calculated first. Index scores are scores which reflect a performance score for a particular skill or for a particular cognitive domain. Thus, index scores can be calculated for particular tests 24 by algorithmically combining outcomes from outcome parameters 28 of the test 24 into a unified score. This algorithmic combination may be linear, non-linear, or any type of arithmetic combination of scores. For example, an average or a weighted average of outcome parameters may be calculated. Alternatively, index scores can be calculated for particular cognitive domains from multiple data sources by algorithmically combining outcomes from selected outcome parameters 28 within the cognitive domain 26. This algorithmic combination may be linear, non-linear, or any type of arithmetic combination of scores. For example, an average or a weighted average of outcome parameters may be calculated. The calculation of index scores continues until all selected data has been processed. At this point, the calculated index scores are either sent (step 114) directly to reporting module 22, or alternatively, processor 20 calculates (step 116) a composite score, and sends (step 114) the composite score to reporting module 22. In one embodiment, there is no index score calculation at all, and processor uses the selected data to directly calculate (step 116) a composite score. In some embodiments, the composite score further includes input from data which is collected (step 118) from other data sources, such as, for example, background data source 14, and/or medical data source 16.

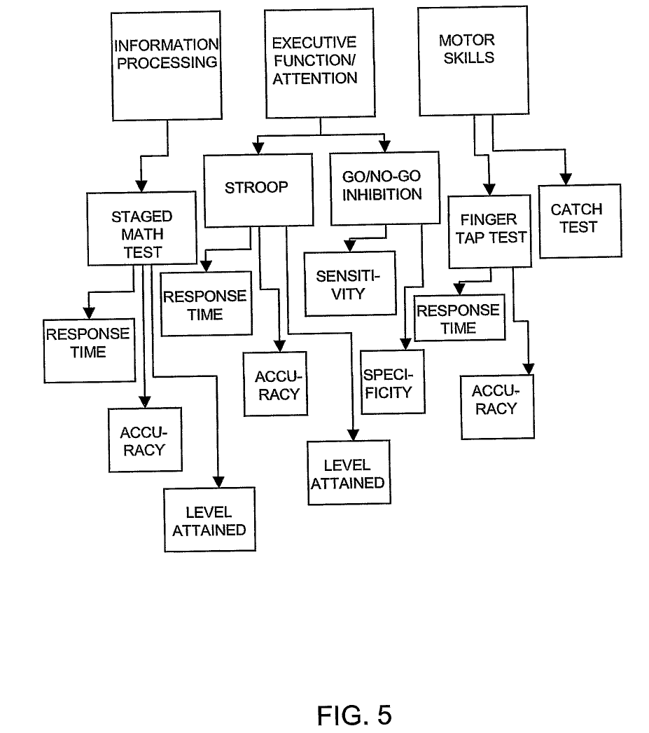

Reference is now made to FIG. 5, which is a schematic illustration of one specific example of the multi-layered collection of data generally depicted in the schematic illustration of FIG. 2. In the embodiment shown in FIG. 5, the cognitive domains of information processing, executive function/attention, and motor skills are selected. A staged math test is used for information processing; a stroop test and a Go/NoGo Inhibition test are used for executive function/attention; and a finger tap test and a catch test are used for motor skills. Specific details about each of these tests are described in the '380 Publication. As disclosed in the '380 Publication, each cognitive test includes several levels, practice sessions, layers of data, quality assurance, and many other features. Specific outcome parameters, such as response time, accuracy, level attained, etc. are collected and processed.

### Staged Math Test

As described in the '380 Publication, the staged math test is designed to assess a subject's ability to process information, testing both reaction time and accuracy. Additionally, this test evaluates math ability, attention, and mental flexibility, while controlling for motor ability.

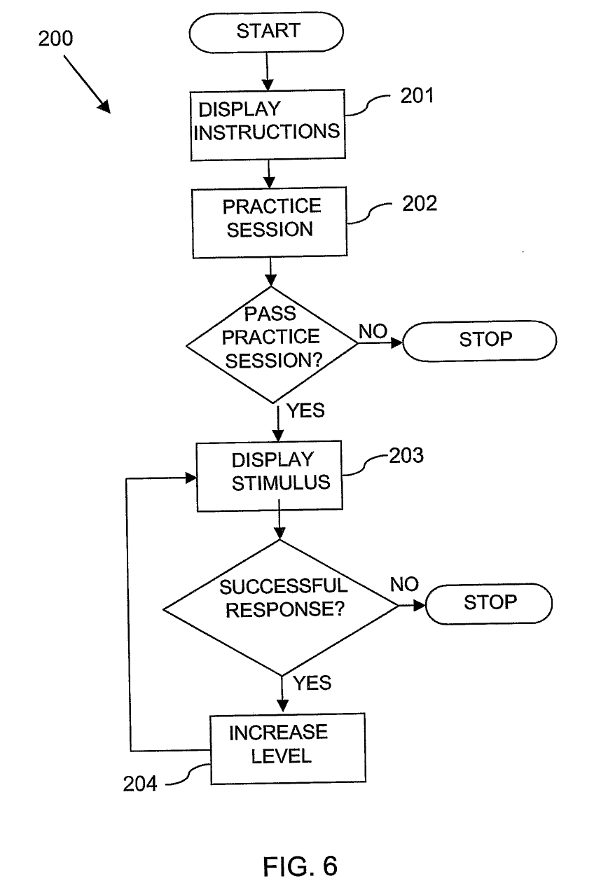

Reference is now made to FIG. 6, which is a flow chart diagram illustration of the steps of a test 200. In a preferred embodiment, the test consists of at least three basic levels of difficulty, each of which is subdivided into subsection levels of speed. The test begins with a display of instructions (step 201) and a practice session (step 202). The first subsection level of the first level is a practice session, to familiarize the subject with the appropriate buttons to press when a particular number is given. For example, the subject is told that if the number is 4 or less, he/she should press the left mouse button. If the number is higher than 4, he/she should press the right mouse button. The instructions continue with more detailed explanation, explaining that if the number is 4, the subject should press the left mouse button and if the number is 5, the subject should press the right mouse button. It should be readily apparent that any number can be used, and as such, the description herein is by way of example only.

A number is then shown on the screen. If the subject presses the correct mouse button, the system responds positively to let the user know that the correct method is being used. If the user presses an incorrect mouse button, the system provides feedback explaining the rules again. This level continues for a predetermined number of trials, after which the system evaluates performance. If, for example, 4 out of 5 answers are correct, the system moves on to the next level. If less than that number is correct, the practice level is repeated, and then reevaluated. If after a specified number of practice sessions the performance level is still less than a cutoff percentage (for example, 75% or 80%), the test is terminated.

The test is then performed at various levels, in which a stimulus is displayed (step 203), responses are evaluated, and the test is either terminated or the level is increased (step 204). The next three subsection levels perform the same quiz as the trial session, but at increasing speeds and without feedback to the subject. The speed of testing is increased as the levels increase by decreasing the length of time that the stimulus is provided. In all three subsection levels, the duration between stimuli remains the same.

The next level of testing involves solving an arithmetic problem. The subject is told to solve the problem as quickly as possible, and to press the appropriate mouse button based on the answer to the arithmetic problem. For the example described above, if the answer to the problem is 4 or less, the subject must press the left mouse button, while if the answer to the problem is greater than 4, the subject must press the right mouse button. The arithmetic problem is a simple addition or subtraction of single digits. As before, each set of stimuli is shown for a certain amount of time at the first subsection level and subsequently decreased (thus increasing speed necessary reaction time) at each further level.

The third level of testing is similar to the second level, but with a more complicated arithmetic problem. For example, two operators and three digits may be used. After each level of testing, accuracy is evaluated. If accuracy is less than a predetermined percentage (for example, 70%) at any level, then that portion of the test is terminated. It may be readily understood that additional levels are possible, both in terms of difficulty of the arithmetic problem and in terms of speed of response.

It should be noted that the mathematical problems are designed to be simple and relatively uniform in the dimension of complexity. The simplicity is required so that the test scores are not highly influenced by general mathematical ability. In one embodiment, the stimuli are also designed to be in large font, so that the test scores are not highly influenced by visual acuity. In addition, since each level also has various speeds, the test has an automatic control for motor ability.

The system collects data regarding the response times, accuracy and level reached, and calculates scores based on the collected data.

### Stroop Test

A Stroop test is a well-known test designed to test higher brain functioning. In this type of test, a subject is required to distinguish between two aspects of a stimulus. In the Stroop test described in the '380 Publication, the subject is shown words having the meaning of specific colors written in colors other than the ones indicated by the meaning of the words. For example, the word RED is written in blue. The subject is required to distinguish between the two aspects of the stimulus by selecting a colored box either according to the meaning of the word or according to the color the word is written in. The additional parameter of speed is measured simultaneously.

The first part of the test is a practice session. The system displays two colored boxes and asks the subject to select one of them, identifying it by color. Selection of the appropriate box may be accomplished by clicking the right or left mouse button, or by any other suitable method. The boxes remain visible until a selection is made. After responding, the system provides feedback if the incorrect answer was chosen. The practice session is repeated several times. If the performance is less than a predetermined percentage (for example, 75% or 80%), the practice session is repeated. If it is still less than the predetermined percentage after another trial, then the test may be terminated.

Once the practice session is completed, the system presents a random word written in a certain color. In addition, the system presents two boxes, one of which is the same color as the word. The subject is required to select the box corresponding to the color of the word and is not presented with feedback. This test is repeated several times. On the next level, the system presents the words “GREEN”, “BLUE” or “RED”, or another word representing a color. The word is presented in white font, and the system concurrently presents two boxes, one of which is colored corresponding to the word. The subject is required to select the box corresponding to the color related to the meaning of the word without receiving feedback. This test is repeated several times, preferably at least 2-3 times the number of samples as the first part. In this way, the subject gets used to this particular activity.

The next level is another practice session, in which the system presents a color word written in a color other than the one represented by the meaning of the word. The subject is instructed to respond to the color in which the word is written. Because it is a practice session, there is feedback. The test is repeated several times, and if the performance is not above a certain level, the test is terminated. If the subject is successful in choosing the color that the word is written in rather than the color that represents the meaning of the word, the next level is introduced.

The next level is the actual “Stroop” test, in which the system displays a color word written in a color other than the one represented by the word. The word is visible together with two options, one of which represents the color the word is written in. The subject is required to choose that option. This test is repeated numerous times (30, for example), and there is no feedback given. Level, accuracy and response time are all collected and analyzed.

### Go/NoGo Response Inhibition

As described in the '380 Publication, a Go/No Go Response Inhibition test is provided in accordance with one embodiment of the present invention. The purpose of the test is to evaluate concentration, attention span, and the ability to suppress inappropriate responses.

The first level is a practice session. The system displays a colored object, such as a box or some other shape. The object is a single color, preferably red, white, blue or green. It should be noted that by using a color as a stimulus, rather than a word such as is the case in prior art tests of this type, the test is simplified. This simplification allows for subjects on many different functional levels to be tested, and minimizes the effect of reading ability or vision. The subject is required to quickly select a mouse button for the presence of a particular color or not press the button for a different color. For example, if the object is blue, white or green, the subject should quickly press the button, and if the object is red, the subject should refrain from pressing the button. It should be readily apparent that any combination of colors may be used.

The first level of the test is a practice session, wherein the subject is asked to either react or withhold a reaction based on a stimulus. Each stimulus remains visible for a predetermined amount of time, and the subject is considered to be reactive if the response is made before the stimulus is withdrawn. In a preferred embodiment, the system presents two red objects and two different colored objects, one at a time, each for a specific amount of time (such as a few hundred milliseconds, for example). The subject is asked to quickly press any mouse button when any color other than red is displayed, and to not press any button when a red color is displayed. Feedback is provided in between each of the trials to allow the user to know whether he/she is performing correctly. If the subject has at least a certain percentage correct, he/she moves on to the next level. Otherwise, he/she is given one more chance at a practice round, after which the test continues or is terminated, depending on the subject's performance.

There may be only one testing level for this particular embodiment, in which the stimuli are similar to the ones given in the practice session, but the subject is not provided with any feedback. Both sensitivity and specificity are calculated.

### Finger Tap Test

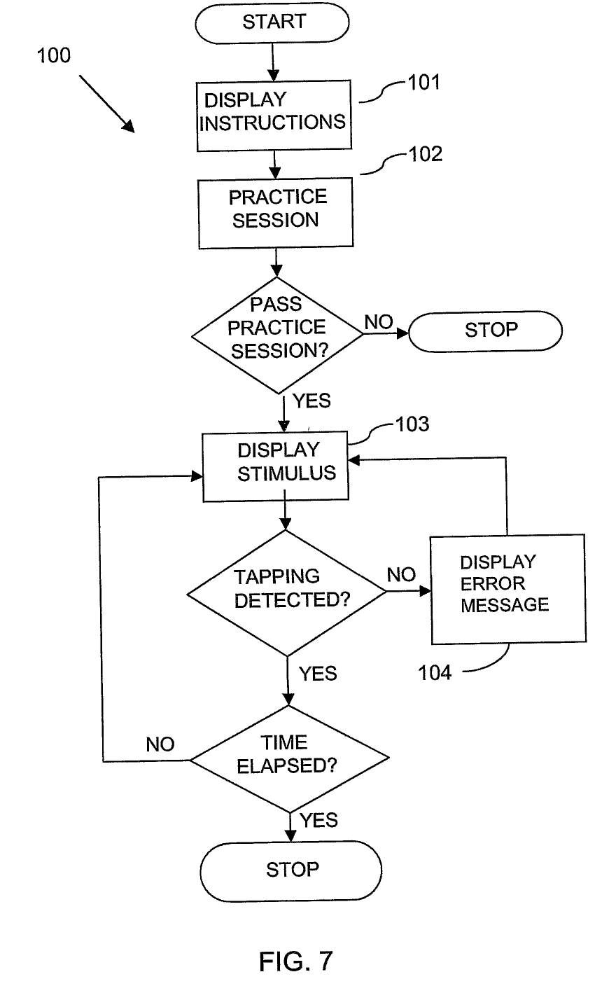

As described in the '380 Publication, a finger tap test is designed to assess speed of tapping and regularity of finger movement. Reference is now made to FIG. 7, which is a flow chart diagram illustration of the steps of a finger tap test according to one embodiment of the present invention. At the beginning of the test, the system displays (step 101) instructions. The instructions describe what the subject will see on the screen, and instruct him/her what to do when the stimulus appears. The message may be very detailed, specifying, for example, which hand to use. The subject is asked to tap in response to a specific stimulus. Initially, the system runs a practice session (step 102), in which a very basic form of the test is given, along with feedback informing the subject whether or not the test is being done properly. The subject is given several chances to perform the requested task, and if the initial score is below a certain predetermined level, the test is terminated. In a preferred embodiment, the scoring is designed to elucidate whether or not tapping was detected. If it was detected a certain percentage of time, the test continues.

The main testing portion begins by displaying (step 103) a stimulus for a predetermined amount of time. In a preferred embodiment, the stimulus is a bar or line on the screen which increases in length with time. In alternative embodiments, the stimulus is a shape which moves across the screen, or is any other form and movement which is displayed for a predetermined amount of time. In one embodiment, the predetermined amount of time is 10-15 seconds. In a preferred embodiment, the stimulus is displayed for 12 seconds. It should be readily apparent that the stimulus may be displayed for any length of time which may be useful in testing the response. The subject is expected to repeatedly tap as quickly as possible in response to the stimulus, as explained in the instructions or by a test administrator prior to commencement of the testing portion. In a preferred embodiment, tapping is done on one of the mouse buttons. Alternative embodiments include tapping on a finger pad, a keypad, or any other button or object configured to convert mechanical input (tapping) to electrical signals, which are then sent to a processor.

If tapping is detected, data is collected during the time it takes for the stimulus to move across the screen, or until some other indication is made to stop. If tapping is not detected, the system displays (step 104) an error message, after which the stimulus is displayed again. The error message may be a reminder of how to respond. If tapping is detected, the test continues until the predetermined amount of time has elapsed. Once the time has elapsed, the test ends.

Detection of tapping is determined by specific criteria. For testing purposes, tapping is considered to not have occurred if the inter-tap interval, or ITI, is greater than a predetermined amount.

Once the testing sequence is completed, outcome is determined based on several parameters, including the times at which the test began and at which the response was received, the overall mean and standard deviation of ITI for right hand and for left hand (i.e. a measure of the rhythmicity of the tapping), and the number of taps per session.

### Catch Test

A second example of a test which may be included in a battery is a catch test, also designed to test motor skills. As described in the '380 Publication, the catch test is designed to assess hand/eye coordination, speed of movement, motor planning, and spatial perception.

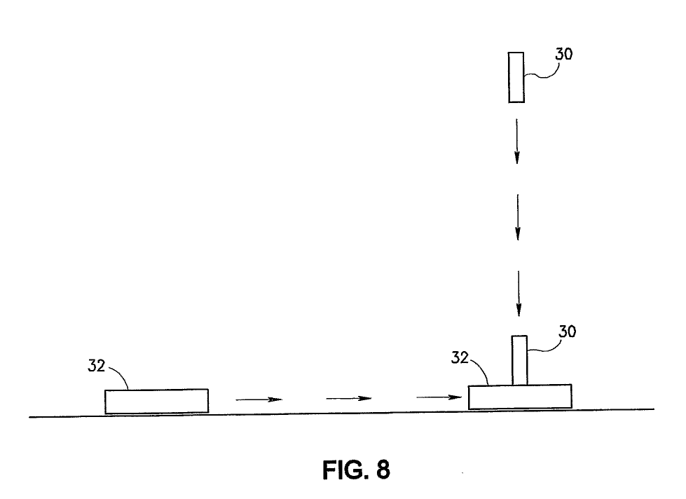

Reference is now made again to FIG. 6 and to FIG. 8, which depict a flow diagram of the steps of a test 200, and a sample screen shot of a catch test in session, according to one embodiment of the present invention. The subject is asked to catch a first object 30 falling from the top of a screen using a second object 32 on the bottom of the screen, as shown in FIG. 8 and described in further detail hereinbelow. An important aspect of this test is that its simplicity allows for a very short learning curve, thereby minimizing effects of prior computer use on test performance. That is, a person with little or no experience is able to perform comparably with a person with a great deal of computer experience within a very short time, thereby allowing for isolation of the particular skills to be tested.

First, the system displays (step 201) a set of instructions. The instructions direct the subject to catch the falling object with a movable object on the bottom of the screen. In a preferred embodiment, the falling object 30 is a simple shape and color, such as a green square or a blue ball. In a preferred embodiment, the movable object 32 is a straight line or some other simple shape that might represent a paddle or racquet, such as the shape depicted in FIG. 8. It should be readily apparent that any suitable shape may be used, including more complex configurations such as sports items (i.e., baseball and glove), space items (i.e., aliens falling and a force shield on the bottom), or any other suitable combination. In the instructions, the subject is directed as to how to move object 32 from side to side. Any button may be configured to allow object 32 to move in a controlled manner. In a preferred embodiment, the right mouse button may be used to move object 32 to the right and the left mouse button to move object 32 to the left, or arrow buttons on a keyboard may be used. In a preferred embodiment, each mouse click moves the object one length, and the object cannot leave the bounds of the screen. However, it should be readily apparent that the control mechanism is not limited to those listed herein, and any suitable control mechanism may be used.

The test begins by providing (step 202) a practice session. In the practice session, the subject is expected to catch a falling object. If the subject catches the object, the system displays a positive feedback message. If the subject does not catch the element, the system displays a feedback message explaining that the objective is to catch the object falling from the top of the screen, and further explaining how to move the object. Once a predetermined number of trials are successfully completed, the test moves on to the next level. Successful completion of the practice session is determined by a percentage of successful catching of the object. In a preferred embodiment, the subject must catch the object at least 2 out of 3 times in order for the testing session to continue.

If the practice session is passed, the test continues by displaying (step 203) the falling object 30 at a predetermined speed and calculating the number of successful catches. If the catching score is higher than a predetermined level, the test continues by moving onto the next level, at which object 30 is configured to fall at a faster speed. If the catching score is lower than the predetermined level, the testing session is terminated.

Subsequent levels each have a faster falling rate than the previous level. It should be readily apparent that any time interval may be used, as long as each level has a faster rate than the previous one. In addition, any number of levels may be used, until the subject reaches a point at which the test is too difficult.

The starting position of both the falling object 30 and the movable object 32 in relation to the falling element vary from trial to trial. In addition, the path of falling object 30 is also variable, and may be useful in increasing the difficulty of the test. For all levels, if the subject performs a successful catch a predetermined number of times, the test moves on to the next level. Otherwise, the test is terminated.

The system collects data related to the responses, including timing, initial location of element and object, number of errors, number of moves to the left and to the right, and level of testing, and presents a score or multiple scores based on the above parameters.

Once the tests are administered and data is collected, data selector 38 selects outcome parameters for data calculation. For example, data selector 38 may select response times from the staged math test and the stroop test, accuracy for all of the tests, speed for the finger tap test, and number of errors and number of moves for the catch test. As another example, data selector 38 may select all of the outcome parameters from all of the tests. Any combination may be selected, and the selection may either be pre-programmed, may depend on other collected data from the same individual or from published information, or may be manually selected.

It should be readily apparent that other batteries of tests for other cognitive domains may be used. For example, tests for verbal or non-verbal memory may be used for the memory domain (to exclude Alzheimer's, for example), or cognitive tests which include a measure of visual/spatial orientation may be included. For certain applications, the emphasis can be placed on one or two particular cognitive domains. In other embodiments, a comprehensive testing scheme may be administered, taking into account many cognitive domains. Comparisons of various domains can give an indication that one condition is likely or that another condition can definitely be excluded. For example, a relatively more severe executive function deficit may indicate Parkinson's while a relatively more severe memory deficit may indicate Alzheimer's.

All tests in the battery may provide a wide range of testing levels, practice sessions to eliminate the bottom portion of the learning curve, unbiased interaction between the patient and clinician, and a rich amount of data from which to calculate scores.

### Background Data Source

Background data source 14 may include a questionnaire with questions about disease duration, profile of symptoms, side effects of medication, performance while on and off medication, history, personal information, questions related to anxiety level and/or mood, questions related to activities of daily living (ADL)—including driving, shopping, ability to manage finances, household chores, and the like. Answers may be yes/no answers, or may be graded responses, such as rating on a scale of 1-10.

### Medical Data Source

Medical data source 16 may include a medical history of the individual to be tested (ie, official medical records), and a questionnaire including questions regarding medication response, presence of non-Parkinson's indications, clinical findings, and general cognitive and motor function. Such forms may also include scoring for each type of questions, which may or may not be incorporated into the scoring algorithm of the system of the present invention.

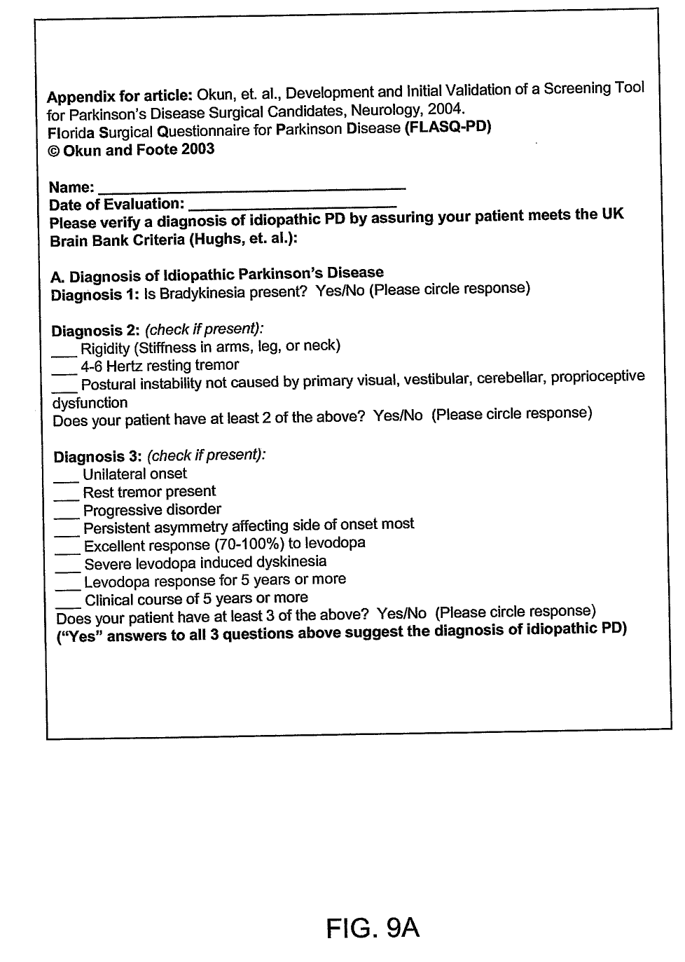

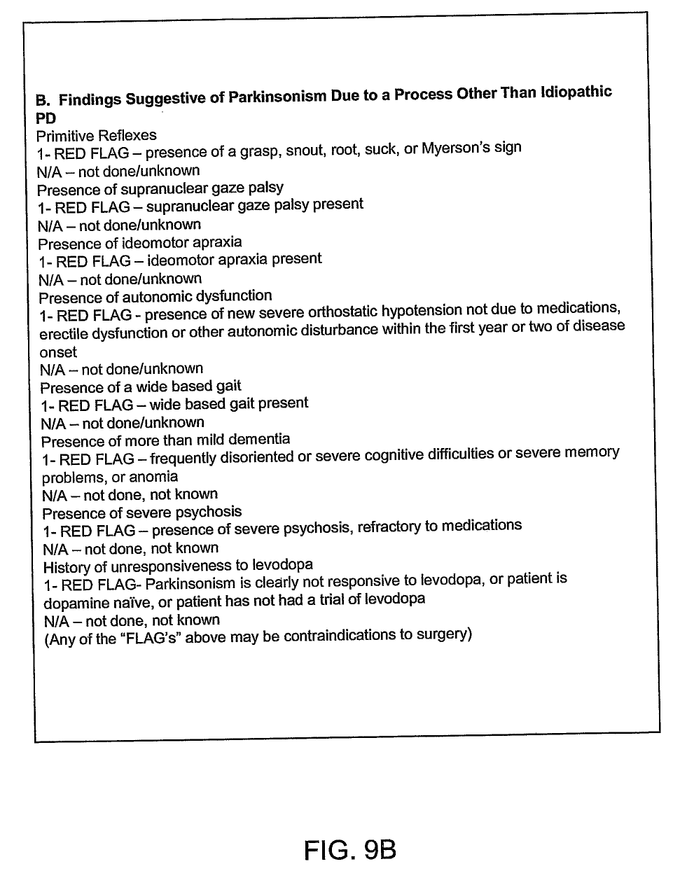

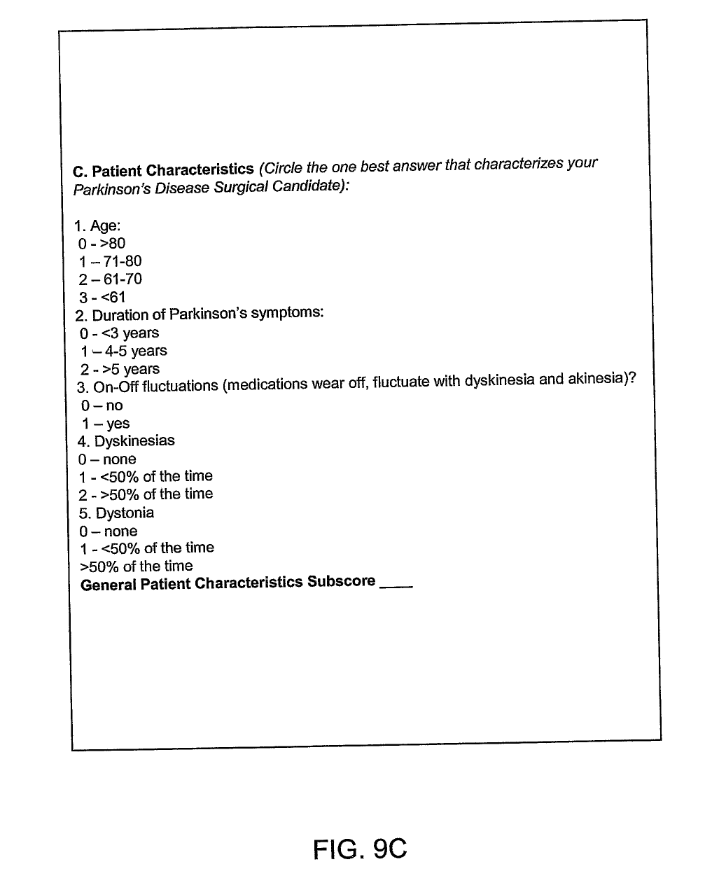

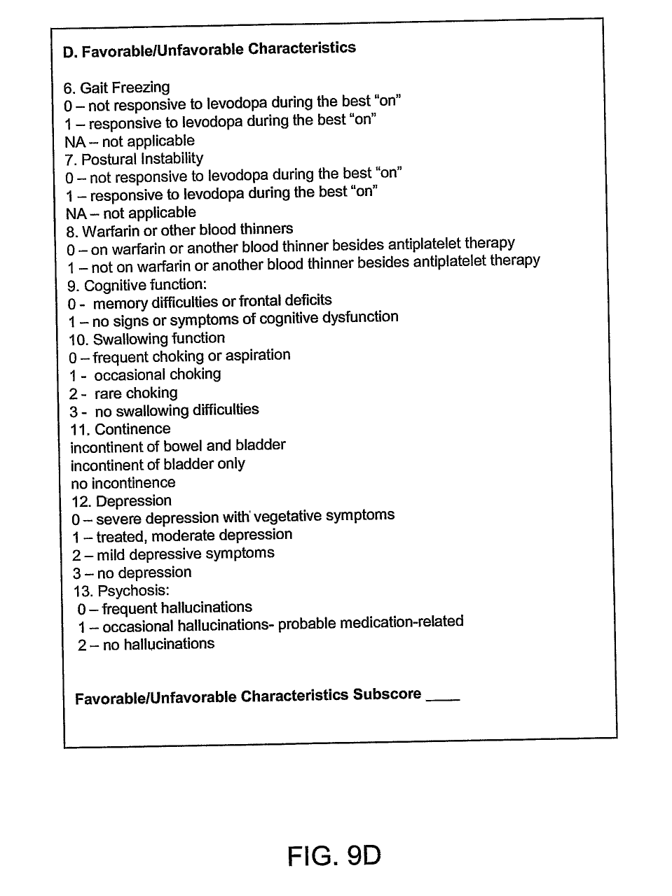

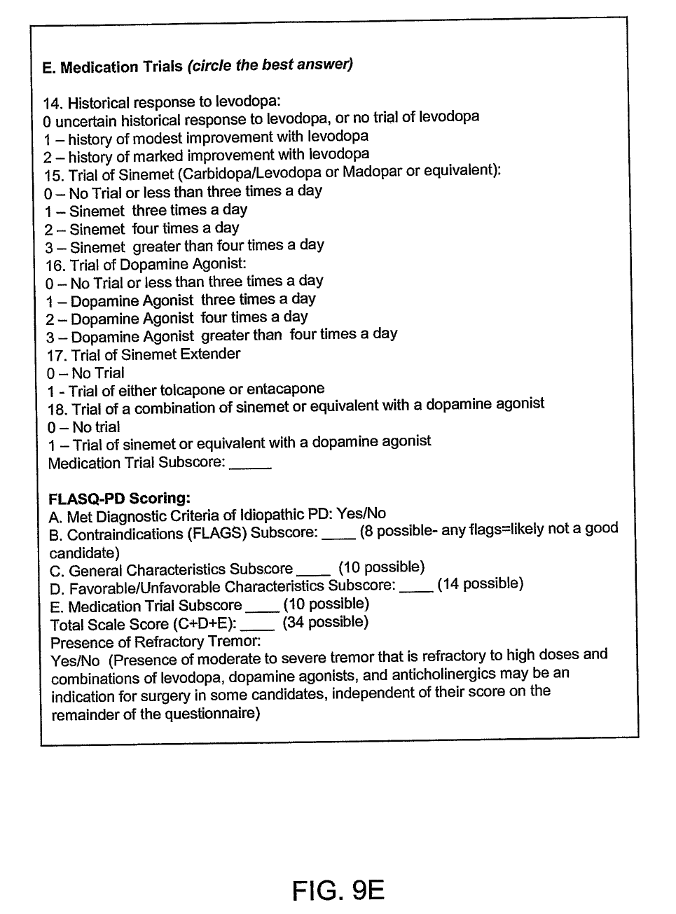

One particular questionnaire or form that has been developed for the screening for DBS is the Florida Surgical Questionnaire for Parkinson Disease (FLASQ-PD), discussed more fully in Okun et al., Development and Initial Validation of a Screening Tool for Parkinson's Disease Surgical Candidates, Neurology, 2004, incorporated herein by reference in its entirety. A copy of an example of a FLASQ-PD is included as FIGS. 9A-9E. Briefly, the FLASQ-PD is a five-part questionnaire. The first part tests for a diagnosis of idiopathic PD. Questions related to the presence of bradykinesia, rigidity, resting tremor, postural instability, asymmetry, response to levodopa, and clinical course, for example, are presented. The second part tests for particular “red flags” which are suggestive of non-idiopathic PD. The third part collects information about general patient characteristics, such as age, duration of symptoms, response to medication, dyskinesias and dystonia. The fourth part tests for favorable or unfavorable characteristics, such as gait, postural instability, presence of blood thinners, cognitive function, depression, psychosis, incontinence, swallowing difficulties, etc. The fifth part details a history of medication trials. Each of the five parts has a subscore, which can then be combined to provide an overall score for candidacy based on the questionnaire.

The questionnaires for background data source 14 and/or medical data source 16 may be completed by the individual, or by a person close to the individual, such as a family member, with or without input from the individual as well. When appropriate (such as with the FLASQ-PD), questionnaires are filled out by a clinician. In one embodiment, questionnaires are presented via the computer, and the answers to the posed questions are stored in a database. Alternatively, the questionnaires are presented on paper, and the answers are later entered into a computer.

### Anxiety/Depression Data Source

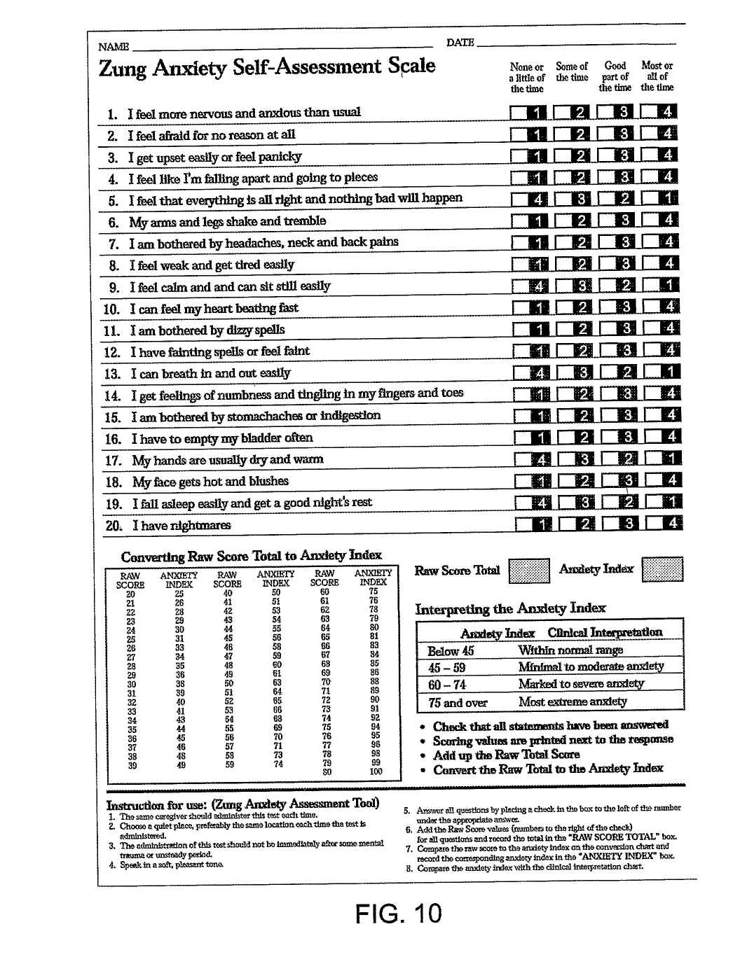

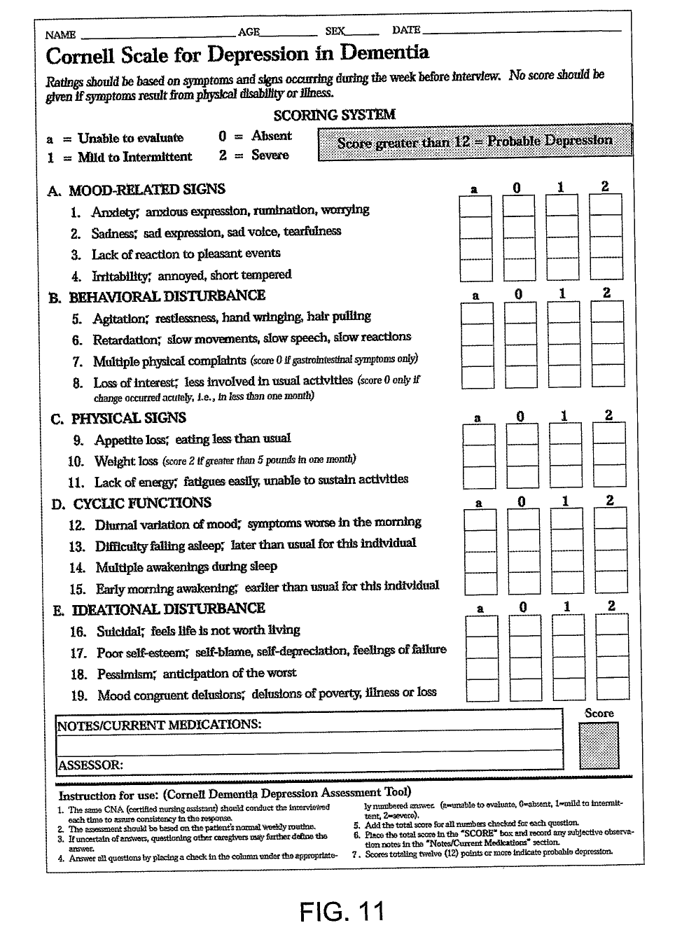

Anxiety/depression data source 18 includes tests for anxiety and for depression, either one of which the presence of would be a contraindication to surgery. Known scales for measuring anxiety and separate scales for measuring depression are used. For example, the Zung Anxiety Self-Assessment Scale, a copy of which is attached hereto as FIG. 10, is a scale which includes questions about nervousness, dizziness, sleeping abilities, physical discomforts, etc and determines a score for anxiety based on a patient's response to the various questions. Other known scales which may be used as an anxiety data source for the purposes of the present invention include the Hamilton Anxiety Scale, the Sheehan Patient Rated Anxiety Scale, the Anxiety Status Inventory, and any other known scales for measuring anxiety and providing a score. An example of a known scale for measuring depression includes the Cornell Scale for Depression in Dementia, a copy of which is attached hereto as FIG. 11. This scale includes questions about mood, behavior, physical signs of depression, cyclic functions (such as sleep disturbances, or mood changes at different times of day), and ideational disturbances (such as suicidal tendencies, pessimism, delusions, etc.) and determines a score for depression based on a patient's response to the various questions.

### Motor Skills Data Source

Motor skills are evaluated by known methods. For example, motor testing can be assessed using measuring devices for testing for tremor, postural instabilities, balance, muscle strength, coordination, dexterity, and motor learning, for example. Such devices are known, and may include for example, triaxial accelerometers, hand dynamometers, Purdue pegboards, and others. In some embodiments, motor skills are evaluated using cognitive tests, similar to the ones described above or described in the '380 Publication. All response data and/or measured data is collected, and either sent to reporting module 22 or integrated into a composite score with other collected data.

### Data Processing

Responses and/or scores from some or all of data sources 12, 14, 16, 18 and 19 are collected and summarized, or are used to calculate more sophisticated scores such as index scores and/or composite scores. In one embodiment, decision points are included along the way, wherein a particular result or set of results gives a clear indication of candidacy for surgery or for exclusion from candidacy for surgery. For example, if certain “red flags” of the second part of the FLASQ-PD were positive, the candidate could be automatically excluded based on that determination alone. Many other “determinate” points are possible, in each of the domains. Other examples may include a failing score on the anxiety or depression scales (indicating that anxiety and/or depression is present) or general cognitive function in the abnormal zone based on cognitive tests. In addition to individual decision points, a total score which reflects a combination of the different elements of the system is presented as well. Decisions regarding candidacy may stem from one or several of the above elements, depending on the data, the individual, and the physician's requirements. The order of scoring may be interchangeable among each of the elements.

Index scores are generated for each cognitive domain based on the tests and/or results from other data sources. For example, an index score may be generated from a combination of data collected from cognitive outcomes related to motor skills (such as response time, for example) and from measurements of an outcome from motor skills data source, such as tremor. Alternatively, an index score may be generated for a particular domain based only on cognitive test responses. The index score is an arithmetic combination of several selected normalized scores. This type of score is more robust than a single measure since it is less influenced by spurious performance on any individual test. For example, an executive function index score may be comprised of individual measures from a Stroop test and a Go/NoGo Inhibition test. Alternatively, an executive function index score may be comprised of individual measures from one test (such as a Stroop test) over several trials. An example of an algorithm for computing the index score, according to one preferred embodiment, is a linear combination of a specific set of measures. The selection of the member of the set of measures and the weighting of each member is based on the known statistical method of factor analysis. The resulting linear combination is then converted to an index score by calculating a weighted average.

Composite scores may be calculated based on data from several index scores and may further be combined with specific scores from the additional data sources (i.e., background or medical data source, motor skills source, etc.) to provide a comprehensive candidacy score. In alternative embodiments, composite scores may be calculated based on a combination of one index score and specific scores from the additional data sources. In yet another embodiment, composite scores may be calculated from particularly selected normalized outcome measures, and may further be combined with data from the additional data sources.

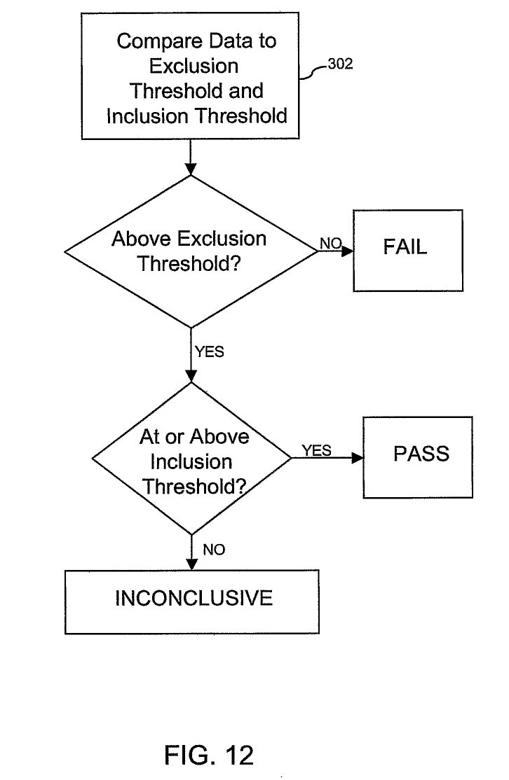

Reference is now made to FIG. 12, which is a flow chart diagram of a method of providing a designation for a particular test based on the results of that test. Each of data sources 12, 14, 16, 18 and 19 may have an internal algorithm which allows for designations of “pass” (i.e., patient is a good surgical candidate), “fail” (i.e., patient is not a good surgical candidate at this time) or “inconclusive” (i.e., further evaluation is needed). It should be readily apparent that these terms are to be taken as representative of any similar terms to be used in the same context, such as, for example, “threshold reached”, “maybe pass”, “undetermined”, “currently good candidate”, “yes”, “no” or the like. Processor 20 first compares (step 302) data from a particular source to a pre-defined threshold value for inclusion and a pre-defined threshold value for exclusion. Alternatively, the pre-defined threshold values may each include several threshold values or ranges of values. If the data is not above the exclusion threshold value, the result for the particular test is “fail.” If the data is above the exclusion threshold value, it is compared to the inclusion threshold value. If it is above the inclusion threshold value, the result is “pass.” If it is not above the inclusion threshold value, the result is “inconclusive.” The data which is used for the comparison may be, for example, a final score for the particular test, after all data has been evaluated. This final score may be a single test score or an index score compiled from multiple tests, either within the same cognitive domain or from several cognitive domains. Alternatively, the data may be compared to the threshold values at the outcome measure level, wherein the comparison includes separate comparisons for each of the outcome measures for the specific test. In this case, it may be determined, for example, that if all outcome measures are below the exclusion threshold, or if a certain percentage of the outcome measures are below the exclusion threshold the result is “fail”. If all outcome measures are above the inclusion threshold, or if a certain percentage of the outcome measures are above the inclusion threshold, the result is “pass.” Otherwise, the result is “inconclusive.”

Examples of thresholds for particular tests include, for example, the following. For the FLASQ-PD, each part may have individual Pass/Fail/Inconclusive designations. For example, a “pass” designation may be given for the first part if the responses to all questions were “yes”, “fail” if the response to the first question was “no” or if the response to both the second and third questions were “no”, and “inconclusive” if the response to either of questions two or three is “no.” For the second part, if the only red flag is for primitive reflexes, or if there were no red flags, the designation may be “pass”, if one red flag was indicated (other than for primitive reflexes), the designation may be “inconclusive”, and if 3 or more red flags or a red flag for dementia or psychosis were indicated, the designation may be “fail”. For the third part, “pass” would be designated for a score of 7 or greater, “fail” for a score of 2 or less, and “inconclusive” for scores of 3-6 or for a response of “no” to a question regarding on/off fluctuations. For the fourth part, “pass” may be designated for a score of 11 or greater, “fail” for a score below 7 or for an answer of “severe depression with vegetative symptoms” for a question on the presence of depression, and “inconclusive” for a score of 7-10, or for high indications of problems with blood thinners, cognitive function, or psychosis. For the fifth part, a designation of “pass” might be made for a score of 8 or higher, “fail” for a score below 2, and “inconclusive” for a score of 3-7. It should be readily apparent that the above designations are listed for illustrative purposes only, and that many alternative conditions for the designations of each section are possible and fall within the scope and spirit of the present invention. Once designations are obtained for each of the five sections, an overall designation for the FLASQ-PD may be made. For example, if all parts were designated “pass”, the overall FLASQ-PD designation may be “pass”. If any one part was designated “fail”, the overall FLASQ-PD designation may be “fail”. If at least one section was designated “inconclusive”, the overall designation may be “inconclusive”.

For the Zung Anxiety scale, “pass” may be indicated for a score of 44 or below, “fail” for a score of 60 and above, and “inconclusive for score of 45-59, or for certain specific answers (such as frequent dizzy spells or fainting spells, for example). For the Depression scale, “pass” may be indicated for a score of 8 or lower, “fail” for a score of 20-30, and “inconclusive” for a score of 9-19 or for a high score on specific questions (such as lack of energy/fatigue, or diurnal variations of mood). For background data, “pass” may be designated for certain responses and “inconclusive” for other responses. Cognitive history may be designated according to past diagnoses. For example, a past diagnosis of Alzheimer's may be designated “fail”, no cognitive complaints or abnormal findings may be designated “pass”, and a diagnosis of mild cognitive impairment (MCI) may be designated “inconclusive.”

For cognitive tests, there may be various ranges of performance evaluation. Descriptions of such ranges are included in U.S. Patent Publication Number 2005-0187436, incorporated by reference herein in its entirety. Included in that description are ranges of “abnormal”, “normal”, “probable abnormal”, “probable normal”, etc. Thus, it may be determined, for example, that if a global cognitive score is probably normal or normal, a designation of “pass” is given, if the global cognitive score is in the abnormal zone, a designation of “fail” is given, and if the global cognitive score is in the “probable abnormal” zone, a designation of “inconclusive” is given. Alternatively, the designation may be made at the index score level. For example, if at most one index score for one cognitive domain (aside from motor skills, which should be in the abnormal or probable abnormal range for a “pass” designation) is in the “probable abnormal” range, a designation of “pass” may be given. If more than one of the index scores for memory, executive function and attention is in the “abnormal” zone, or if more than two of the index scores for memory, executive function and attention is in the “probable abnormal” zone, or if more than three of any index scores (except motor skills) is in the “probable abnormal” or “abnormal” zone, a designation of “fail” is given. If one of memory, executive function and attention is in the “abnormal” zone or more than one of memory, executive function and attention is the “probable abnormal” zone, or more than two of any index scores (except motor skills) is in the “probable abnormal” or “abnormal” zone, a designation of “inconclusive” may be given. For motor cognitive tests, the designations of “abnormal”, “normal”, “probable normal”, “probable abnormal” etc. would result in an opposite designation for the overall recommendation. That is, if motor skills are abnormal, results are designated as “pass”, since abnormal motor skills might be an indication of Parkinson's Disease. Conversely, if all motor skills are normal, the result would be designated as “fail”, since normal motor skills would contraindicate PD. It should be apparent that many different designations may be defined.

It should be readily apparent that the actual numbers may vary, and that these examples are to be taken as illustrative only. Moreover, designations of fail, pass and inconclusive may be further expanded to include additional designations. For example, a numerical scale may be used, wherein results from each test are listed as a score from 1-5 or 1-10, wherein 1 is the worst result possible, 5 (or 10) is the best result possible, and the additional numbers indicating varying levels in between.

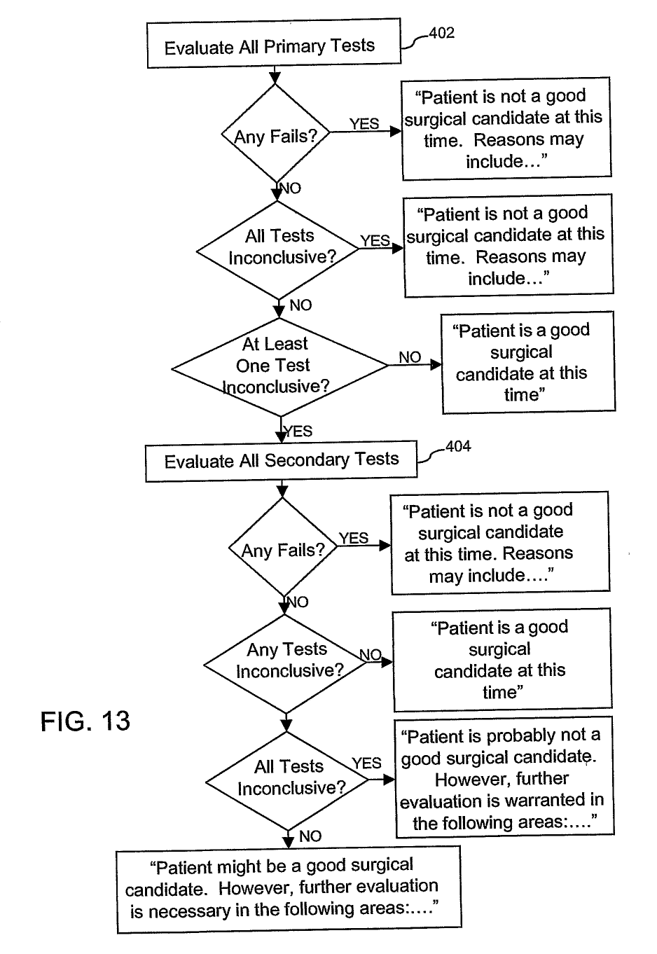

Reference is now made to FIG. 13, which is a flow chart diagram illustration of a method of integrating results from multiple tests from some or all of data sources 12, 14, 16, 18 and 19, in accordance with one embodiment. First, tests are designated as primary tests or as secondary tests. This designation may be pre-determined for particular testing batteries, or may be tailored to an individual. For example, it may be determined that all cognitive tests are primary tests, medical data (such as FLASQ-PD) is a primary test, while background data, anxiety/depression data, and motor skills are secondary tests. Alternatively, it may be determined that particular cognitive tests are primary tests, such as a finger tap test and a catch test, for example, while other cognitive tests are secondary tests. First, processor 20 evaluates (step 402) all primary tests. If any of the primary tests have a “fail” designation, the result is “Patient is not a good surgical candidate at this time. Reasons may include . . . .” Reasons for not including the individual may be given based on which primary tests have failed, and based on specifics about why the failing designation was assigned. If all of the primary tests are “inconclusive”, the result is also “Patient is not a good surgical candidate at this time. Reasons may include . . . .” If some of the tests are not “inconclusive”, the processor checks whether any of the tests are “inconclusive”. If not, that means that all primary tests have been passed and the result is “Patient is a good surgical candidate at this time.” If at least one test is inconclusive, processor 20 evaluates (step 404) all secondary tests. If any of the secondary tests have a “fail” designation, the result is “Patient is not a good surgical candidate at this time. Reasons may include . . . .” If none of the secondary tests have a “fail” designation, the processor checks whether any of the tests are “inconclusive”. If not, all secondary tests have been passed and the result is “Patient is a good surgical candidate at this time.” If at least one of the secondary tests is “inconclusive”, the processor checks whether all of them are “inconclusive”. If they are all “inconclusive”, the result may be “Patient is probably not a good surgical candidate. However, further evaluation is warranted in the following areas: . . . .” If they are not all inconclusive, then some have been passed, and the result is “Patient might be a good surgical candidate. However, further evaluation is necessary in the following areas: . . . .”

It should be readily apparent that many other processes and results are possible. For example, there may be specific designations for results from FLASQ-PD tests, wherein inconclusive designations may result in “May be a surgical candidate under certain conditions” or “Not a good surgical candidate at this time. Reevaluate after medication trial.” Additionally, the criteria for specific results may be different than the ones depicted in FIG. 13 and described with respect thereto. For example, if certain primary tests are inconclusive, the result may be “not a good surgical candidate”, whereas if other primary tests are inconclusive, evaluation of secondary tests may be necessary. Alternatively, it may be decided that if any of the primary tests are inconclusive and any of the secondary tests are inconclusive, the result may be “not a good surgical candidate” or “reevaluate the following skills:” or the like. Any logical progression of integrating the tests from data sources 12, 14, 16, 18 and/or 19 is envisioned and is within the scope of the present invention.

It should be readily apparent that many other processes and results are possible.

### Reporting Module

Index scores and/or composite scores may be graphed in two ways. A first graph shows the score as compared to the general population. The obtained score is shown on the graph within the normal range for the general population. The general population may either be a random sampling of people, or alternatively, may be a selected group based on age, education, socio-economic level, or another factor deemed to be relevant. The second graph shows the score as compared to any previous results obtained from the same battery of tests on the same subject. This longitudinal comparison allows the clinician to immediately see whether there has been an improvement or degradation in performance for each particular index.

Alternatively, the score is calculated and compared to a normal population as well as a disease-specific population, immediately allowing the clinician to see what range the subject's performance fits into. Furthermore, several indices may be compared, so as to determine which index is the most significant, if any. Thus, the practitioner receives a complete picture of the performance of the individual as compared to previous tests as well as compared to the general population, and can immediately discern what type of medical intervention is indicated. It should also be noted that at different points during the test itself, it may be determined that a specific test is not appropriate, and the tests will then be switched for more appropriate ones. In those cases, only the relevant scores are used in the calculation.

Results or designations from the integration method depicted in FIG. 13 may be included in reporting module 22. For example, the report may include index scores, composite scores, graphs, summaries, and a conclusion such as: “Candidate for surgery”, “Further evaluation necessary” or any other result.

Data are processed and compiled in a way which gives the clinician an overview of the results at a glance, while simultaneously including multiple layers of information. Data are accumulated and compiled from the various tests within a testing battery, resulting in a composite score. A report showing results of individual parameters, as well as composite scores is then generated.

The report may be available within a few minutes over the Internet or by any other communication means. The report includes a summary section and a detailed section. In the summary section, scores are reported as normalized for age and educational level and are presented in graphical format, showing where the score fits into pre-defined ranges and sub-ranges of performance. It also includes graphical displays showing longitudinal tracking (scores over a period of time) for repeat testing. Also, the answers given to the questionnaire questions are listed. Finally, it includes a word summary to interpret the testing results in terms of the likelihood of cognitive abnormality and/or the inclusion or exclusion from candidacy for neurosurgery. The detailed section includes further details regarding the orientation and scoring. For example, it includes results for computer orientation for mouse and keyboard use, word reading, picture identification, and color discrimination. Scores are also broken down into raw and normalized scores for each repetition. Thus, a clinician is able to either quickly peruse the summary section or has the option of looking at specific details regarding the scores and breakdown. Each of these sections can also be independently provided. The report further provides a final impression and recommendations. Additionally, the report may include specific recommendations or limitations such as informing the user that the individual should be evaluated further in particular domains, or after a medication trial, for example.

It should be readily apparent that many modifications and additions are possible, all of which fall within the scope of the present invention.

While certain features of the present invention have been illustrated and described herein, many modifications, substitutions, changes, and equivalents may occur to those of ordinary skill in the art. It is, therefore, to be understood that the appended claims are intended to cover all such modifications and changes as fall within the true spirit of the present invention.

1-29. (canceled)

30. A computerized system for evaluating candidacy of a subject for neurosurgery, the system comprising:
a cognitive testing data source, including at least one cognitive test for testing at least one cognitive domain of the subject, said at least one cognitive test providing cognitive data for said at least one cognitive domain;
a medical data source, said medical data source for collecting data relating to indications and contraindications for candidacy for neurosurgery, said medical data source providing medical data;
a processor for integrating said cognitive data and said medical data, said processor comprising a classification algorithm based on expert knowledge about the relevance of said cognitive data and said medical data to the candidacy for neurosurgery, said processor providing an output value for the candidacy of the subject for neurosurgery, said output value based on said classification algorithm; and
a reporting module in communication with said processor and configured to display said output value.

31. The system of claim 30, further comprising an additional data source, said additional data source comprising at least one of: a background data source, an anxiety data source, a depression data source, and a motor skills data source, the additional data source configured to provide additional data for feeding into said classification algorithm to integrate said additional data with said collected cognitive data and said collected medical data.

32. The system of claim 30, wherein said medical data source is a neurosurgical candidacy questionnaire.

33. The system of claim 32, wherein said neurosurgical candidacy questionnaire comprises questions relating to at least one of: a diagnosis of idiopathic Parkinson's disease, a diagnosis of non-idiopathic Parkinson's disease, neurological function, and a history of medication.

34. The system of claim 30, wherein said at least one cognitive test is a battery of computerized tests for testing various cognitive domains.

35. The system of claim 30, wherein said classification algorithm is at least one of: a decision tree algorithm and a flow chart algorithm.

36. The system of claim 30, wherein said classification algorithm includes exclusion thresholds, wherein if individual data points within said cognitive data or said medical data exceed said exclusion thresholds, said output value is a recommendation for non-candidacy.

37. The system of claim 30, wherein said output value is one of: a recommendation for candidacy, a recommendation for non-candidacy, a probable recommendation for candidacy, a probable recommendation for non-candidacy and an inconclusive result.

38. A method of providing a recommendation for candidacy of a subject for neurosurgery, the method comprising:
providing a computerized cognitive test to said subject;
collecting cognitive data, for said subject, from said computerized cognitive test;
providing a medical data source to the subject, the medical data source comprising items relating to indications and contraindications for candidacy for neurosurgery;
collecting medical data, for said subject, from said medical data source; converting said collected cognitive data and collected medical data into a data type suitable for input into a classification algorithm;
integrating said converted cognitive data and said converted medical data, said integrating comprising: inputting said converted cognitive data and said converted medical data into the classification algorithm, and classifying said converted cognitive data and said converted medical data according to the classification algorithm based on inclusion and exclusion criteria for candidacy for neurosurgery, said criteria based on expert knowledge; and
determining a recommendation for candidacy for neurosurgery designation for said subject based on said integration.

39. The method of claim 38, further comprising providing an additional data source to the subject; collecting additional data, for said subject, from said additional data source; converting said additional data into a data type suitable for input into the classification algorithm; and integrating said converted additional data with said converted cognitive data and said converted medical data, wherein said additional data source includes at least one of: a background data source, an anxiety data source, a depression data source, and a motor skills data source.

40. The method of claim 38, wherein said providing a computerized test comprises providing a battery of computerized tests for testing various cognitive domains.

41. The method of claim 38, wherein said converting cognitive data comprises converting the cognitive data into index scores.

42. The method of claim 38, wherein said converting cognitive data comprises comparing said cognitive data to a threshold and providing a cognitive data result based on said comparison.

43. The method of claim 38, wherein said converting medical data comprises providing an overall score for said medical data.

44. The method of claim 38, wherein said converting medical data comprises:
comparing a first part of said medical data to a first threshold and providing a first medical data result based on said first comparison; and
comparing a second part of said medical data to a second threshold and providing a second medical data result based on said second comparison.

45. The method of claim 38, wherein said determining a candidacy designation includes one of: determining that the subject is a candidate, determining that the subject is not a candidate, determining that the subject is probably a candidate, determining that the subject is probably not a candidate, and an inconclusive determination.

46. The method of claim 38, wherein said providing a medical data source to said subject comprises providing a neurosurgical candidacy assessment questionnaire.

47. The method of claim 38, further comprising reporting said candidacy determination in graphical format.

48. A method of providing a recommendation for candidacy of a subject for neurosurgery, the method comprising:
collecting cognitive data from a computerized cognitive test administered to a subject;
calculating a cognitive score for said collected cognitive data;
determining whether said cognitive score is within a cognitive exclusion range;
collecting medical data from a medical data source comprising items with respect to said subject relating to candidacy for neurosurgery;
calculating a medical score from said collected medical data;
determining whether said medical score is within a medical exclusion range;
if the medical score is not within a medical exclusion range and the cognitive score is not within a cognitive exclusion range, then determining a candidacy recommendation based on said cognitive score and said medical score, otherwise recommending the subject for non-candidacy.

49. The method of claim 48, wherein said determining whether said cognitive score is within a cognitive exclusion range comprises:
calculating a second cognitive score;
combining said cognitive score and said second cognitive score into a combined cognitive score; and
determining whether said combined cognitive score is within the cognitive exclusion range.

50. The method of claim 48, wherein said determining whether said medical score is within a medical exclusion range comprises:
calculating a second medical score;
combining said medical score and said second medical score into a combined medical score; and
determining whether said combined medical score is within the cognitive exclusion range.

51. The method of claim 48, wherein said determining a candidacy recommendation based on said cognitive score and said medical score comprises integrating said converted cognitive data and said converted medical data, said integrating comprising:
inputting said converted cognitive data and said converted medical data into the classification algorithm, and
classifying said converted cognitive data and said converted medical data according to the classification algorithm based on inclusion and exclusion criteria for candidacy for neurosurgery,
wherein said inclusion and exclusion criteria are based on expert knowledge.

52. The method of claim 48, wherein said medical data source is a neurosurgical candidacy assessment questionnaire.

53. The method of claim 48, wherein said determining a candidacy recommendation includes one of: determining that the subject is a candidate, determining that the subject is not a candidate, determining that the subject is probably a candidate, determining that the subject is probably not a candidate, and an inconclusive determination.

54. The method of claim 48, further comprising reporting said candidacy determination in graphical format.
